(19) United States
(12) Patent Application Publication
(10) Pub. No.: US 2025/0012709 A1
(43) Pub. Date: Jan. 9, 2025

(54) APPARATUS AND METHOD FOR ANALYSYS OF MEASURED SPECTRUM

(71) Applicant: UCARETRON INC., Anyang-si, Gyeonggi-do (KR)

(72) Inventor: JEE HWAN JANG, Hwaseong-si, Gyeonggi-do (KR)

(73) Assignee: UCARETRON INC., Anyang-si, Gyeonggi-do (KR)

(21) Appl. No.: 18/700,292

(22) PCT Filed: Oct. 12, 2022
(86) PCT No.: PCT/KR2022/015427

§ 371 Date: Apr. 11, 2024

(30) Foreign Application Priority Data
Oct. 12, 2021 (KR) 10-2021-0134729
Nov. 1, 2021 (KR) 10-2021-0148009
Nov. 11, 2021 (KR) 10-2021-0154528

(51) Int. Cl. G01N 21/01; G01N 21/31; G01N 33/66; G06V 10/82

(52) U.S. Cl. CPC G01N 21/01; G01N 21/31; G01N 33/66; G06V 10/82; G01N 2021/0181; G01N 2201/1293; G01N 2201/1296

(57) ABSTRACT

One embodiment of the present invention can provide a method of analyzing a measured spectrum image including the steps of obtaining a measured spectrum image; creating an analysis model by learning the spectrum image with artificial intelligence; and predicting the feature of a test sample of an analyte by inputting the spectrum image of the test sample to the analysis model.

### TECHNOLOGY FIELD

The present invention relates to an apparatus for analyzing a measured spectrum and a method thereof, and more specifically, the present invention relates to an apparatus for analyzing a measured spectrum and a method thereof in which the image of a measurement spectrum measured for the analyte is learned with artificial intelligence (AI), and the feature of the analyte is predicted from the measurement spectrum image of a test sample of the analyte.

### BACKGROUND ART

An artificial intelligence (AI) system is a computer system that implements human-level intelligence, and unlike existing rule-based smart systems, the AI system is a system in which machines learn and make decisions on their own. The more the AI system learns and utilizes appropriate data, the better its recognition rate will be and it will be able to more accurately understand the user's preferences. Accordingly, existing rule-based smart systems are gradually being replaced with machine learning-based artificial intelligence systems.

Prior art literature: Korean registered patent 10-2391934

The above prior art literature relates to an artificial intelligence-based diagnosis system and method for the cancer risk of thyroid nodules in which information on the diagnosis results are generated by analyzing the cancer risk of thyroid nodules in thyroid ultrasound images based on a learned artificial intelligence algorithm and the generated information is provided to the medical staff in charge, thereby reducing unnecessary biopsy processes. The above literature discloses that a data preprocessing apparatus performs preprocessing a received thyroid ultrasound image and generates a learning data set, a verification data set and a test data set.

The prior literature discloses learning actually captured ultrasound images in the artificial intelligence, but does not disclose a technology for learning an electrochemical measurement spectrum.

### SUMMARY OF THE INVENTION

### Technical Problem

In the present invention, it is possible to provide an apparatus for analyzing a measured spectrum and a method thereof in which the image of a measurement spectrum measured for the analyte is learned with an artificial intelligence (AI) and the feature of the analyte is predicted from the measurement spectrum image of a test sample of the analyte.

### Solution to Problem

One embodiment of the present invention may provide a method of analyzing a measured spectrum comprising the steps of obtaining a measured spectrum image, creating an analysis model by learning the spectrum image with an artificial intelligence, and predicting the feature of a test sample of an analyte by inputting the spectrum image of the test sample to the analysis model.

The method of analyzing the measured spectrum may further include preprocessing the spectrum image before creating the analysis model.

The step of preprocessing the spectrum image may include drawing an axis on the spectrum image.

The step of preprocessing the spectrum image may include filling or invert a partial area of the space divided by the spectrum line in the spectrum image.

The step of creating the analysis model may include the steps of feature-mapping the input spectrum image to a convolutional neural network (CNN) and connecting the mapped data to a fully connected layer.

Another embodiment of the present invention may comprise a storage unit for storing a measured spectrum image, an artificial intelligence learning unit for learning the spectrum image stored in the storage unit with an artificial intelligence to thereby create an analysis model, and a spectrum image of a test sample and an analysis unit for predicting the feature of a test sample of an analyte by inputting the spectrum image of the test sample to the analysis model.

The measurement spectrum analysis apparatus may further include an image processing unit for preprocessing the spectrum image before the learning.

The image processing unit may be configured to draw an axis on the spectrum image.

The image processing unit may be configured to fill or invert a portion of the space divided by the spectrum line in the spectrum image.

The learning unit for creating the analysis model may include a mapping unit for feature-mapping the inputted spectrum image to CNN and a classification unit for connecting the mapped data to a fully connected layer.

### Advantageous Effects

According to the present invention, it is possible to learn the image of a measurement spectrum measured for the analyte with an artificial intelligence (AI) and predict the feature of the analyte from the measurement spectrum image of a test sample of the analyte.

### BRIEF DESCRIPTION OF THE DRAWINGS

FIG. 1 is a diagram showing the chronoamperometry technique which is an electrochemical measurement data processing method in existing blood sugar measurement.

FIG. 2 is a diagram showing a cyclic voltammetry which is an electrochemical measurement data processing method in existing blood sugar measurement.

FIG. 3 is a flowchart of a measurement spectrum analysis method according to an embodiment of the present invention.

FIG. 4 is a cyclic voltagram measured using a cyclic voltammetry to measure the sugar in various fruits.

FIG. 5 is a diagram showing an example of an artificial intelligence learning model used in the present embodiment.

FIG. 6 shows the sugar content predicted by the analysis model using the spectrum of the sugar content in the fruits actually measured in FIG. 4 as a test sample.

FIG. 7 shows a preprocessed spectrum image according to the present embodiment.

FIG. 8 shows the sugar content predicted by an analysis model by constructing the analysis model for a plurality of actual fruits and then inputting a test sample into the analysis model.

FIGS. 9 to 18 are diagrams showing an example of preprocessing spectrum images for various measurement spectra.

FIG. 19 is a configuration diagram of a measurement spectrum analysis device according to another embodiment of the present invention.

### DESCRIPTION OF EXEMPLARY EMBODIMENTS

Hereinafter, the present invention will be described in detail with reference to the attached drawings.

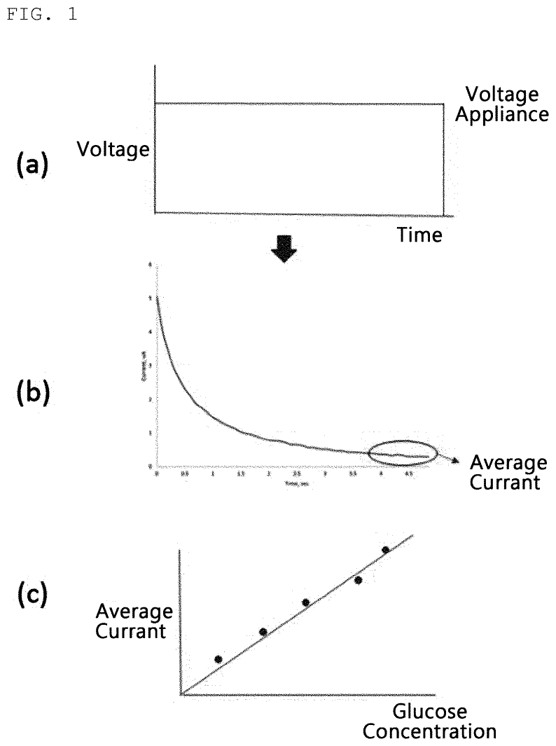

FIG. 1 is a diagram showing the chronoamperometry technique which is an electrochemical measurement data processing method in existing blood sugar measurement.

Referring to FIGS. 1(a) to 1(c), a method is used in which the average current amount is obtained by averaging the current values after the decay curve of the measured current has been reached equilibrium after applying a voltage and which based on this, a calibration equation is obtained and the concentration is calculated by measuring the average current for the unknown glucose concentration. However, there is an irrational aspect in that this requires a highly complicated process of obtaining the average current and calibration equation, a measurement is performed while ignoring a non-faradaic current present in the electrode, information about interfering substances such as solid components or cells present in the analyte may be lost, only a small part of the reaction current curve is used, and so on.

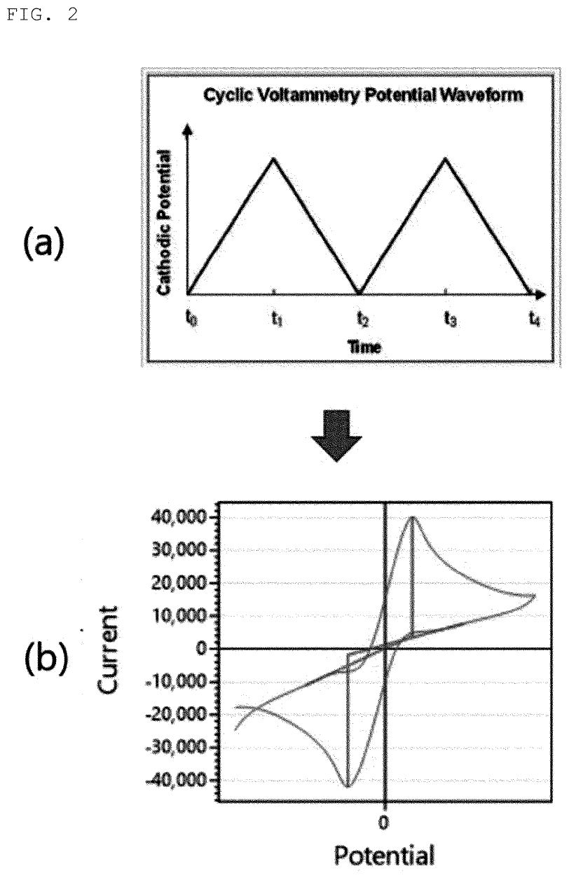

FIG. 2 is a diagram showing a cyclic voltammetry which is an electrochemical measurement data processing method in existing blood sugar measurement.

Referring to FIGS. 2(a) and 2(b), a peak current is defined in such a way of, after applying the voltage while changing over time, obtaining the curve of the measured current, extending the line of the current curve at low voltage, lowering the line from the peak voltage and then calculating the current value up to the point meeting the line. Alternatively, the feature of the cyclic voltagram is extracted and used for analysis. In case that the feature is extracted in this way, much of the information contained in the cyclic voltagram may be lost. For example, in a situation where ionic species do not exist due to insufficient redox reaction, information on the non-faradaic current, etc. may be ignored.

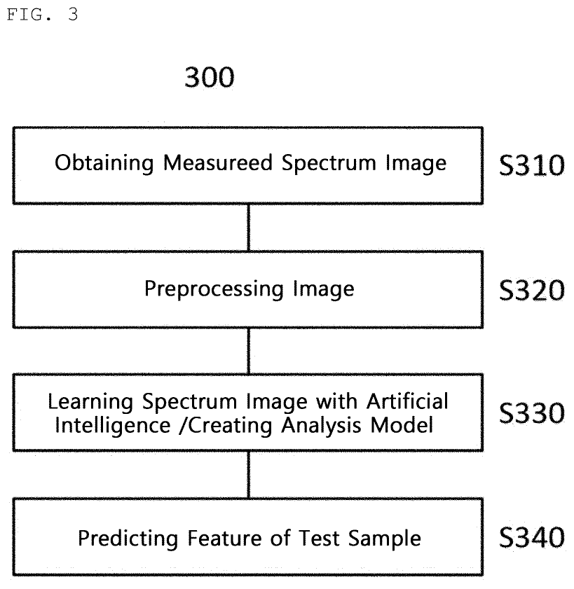

FIG. 3 is a flowchart of a method of analyzing the measurement spectrum according to an embodiment of the present invention.

Referring to FIG. 3, a method of analyzing the measurement spectrum according to the present embodiment may comprise a step 310 of obtaining a measurement spectrum image, a step 330 of creating an analysis model by learning the spectrum image with an artificial intelligence, and a step 340 of predicting the feature of a test sample of the analyte by inputting the spectrum image of the test sample to the analysis model.

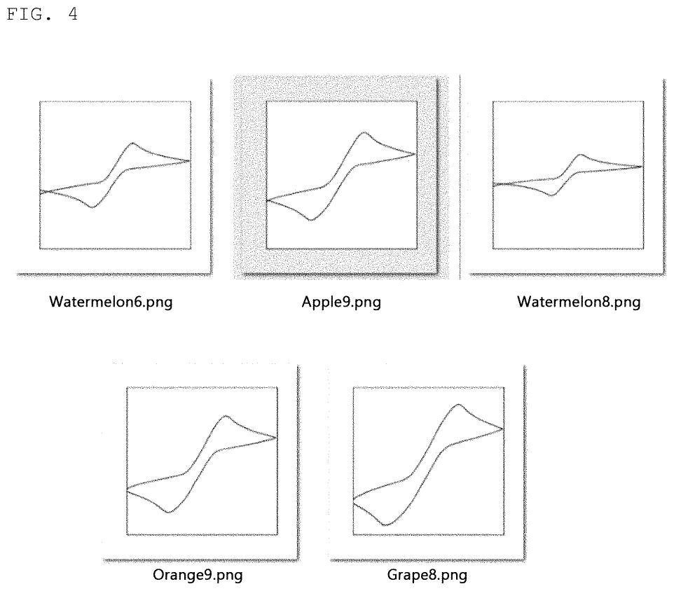

The step 310 of obtaining the measurement spectrum image is a step of obtaining the spectrum for the analyte. In this embodiment, measurement for the analyte may include various measurements, including electrochemical and physical measurements. For example, when measuring a blood sugar in blood, a cyclic voltagram can be obtained using cyclic voltammetry. FIG. 4 is a cyclic voltagram measured using cyclic voltammetry to measure the sugar in various fruits. In FIG. 4, the measurement spectrum image appears as a linear graph forming a closed loop, but the measurement spectrum image may appear in various forms depending on measurement ways or measured parameters. The way of obtaining the spectrum for the analyte in this step is not limited thereto and may be obtained in various ways.

In the step 330 of creating the analysis model, a analysis model may be created by learning the spectrum image with an artificial intelligence. Various machine learning techniques such as PCA analysis, SVM and gradient boosting may be used to learn the spectrum image with the artificial intelligence. In the present embodiment, a convolution neural network (CNN) analysis may be used.

The step of creating the analysis model may include a step of feature-mapping the inputted spectrum image to CNN and a step of connecting the mapped data to a fully connected layer.

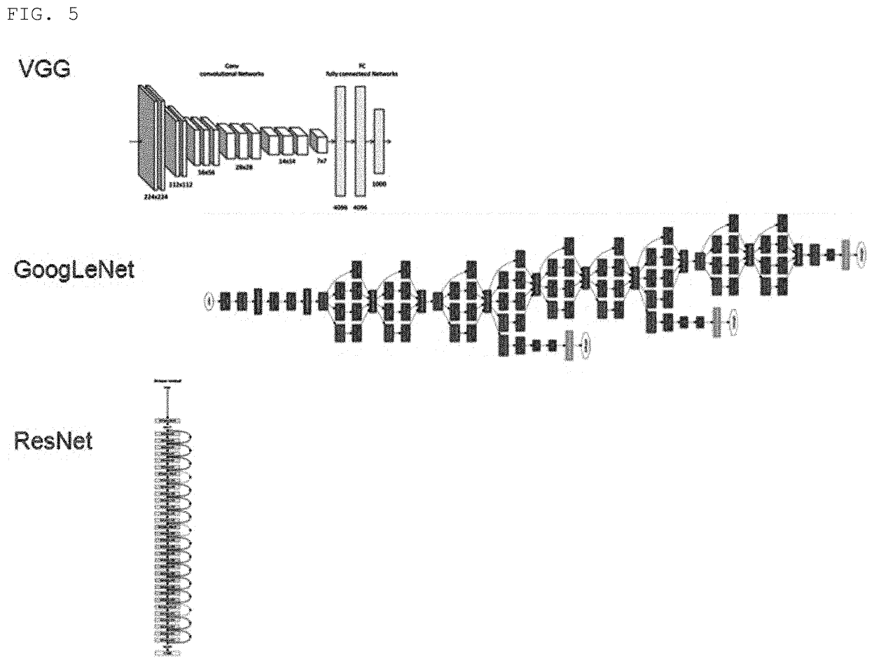

FIG. 5 is a diagram showing an example of an artificial intelligence learning model used in the present embodiment. In this embodiment, the spectrum image may be learned using various CNN structures such as VGG, GoogLeNet, ResNet, LeNet, AlexNet, SENet, VGG-F, VGG-M and VGG-S.

In the step 340 of predicting the feature of the test sample, the feature for the spectrum image of the test sample may be predicted using the analysis model. In this step, the feature of the analyte may be predicted by applying the measurement spectrum for the test sample of the analyte whose feature is to be measured to the analysis model. The feature of the analyte that can be predicted at this step may include various feature, including electrical, chemical and physical feature.

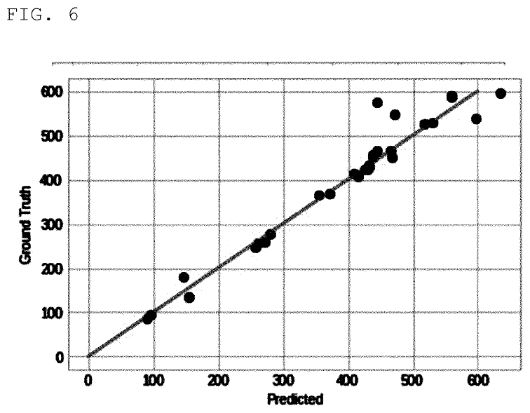

FIG. 6 shows the sugar predicted by the analysis model using the spectrum of the sugar of the fruits actually measured in FIG. 4 as a test sample. Referring to FIG. 6, it can be seen that the sugar (ground truth) measured by the actual blood sugar meter and the sugar (predicted) predicted by the analysis model appear almost similar.

In the present embodiment, a step 320 of preprocessing the spectrum image before creating the analysis model may be further included.

In general, the measured spectrum image can be expressed as a linear graph. When perform a feature mapping by learning the spectrum image with an artificial intelligence, all areas other than the linear area of the spectrum image are recognized as white and thus there is a limit to extracting the feature from the image. The preprocessing of the spectrum image may be performed to compensate for such limitation that may appear during artificial intelligence learning of the measured spectrum image.

The step of preprocessing the spectrum image may be drawing an axis on the spectrum image. The step of preprocessing the spectrum image may be filling or inverting a partial area of the space divided by the spectrum line in the spectrum image. When the spectrum image is preprocessed and then learned with artificial intelligence instead of learning the spectrum image as it is, the learning efficiency for the spectrum image is improved, making it much more efficient to extract the feature within the image.

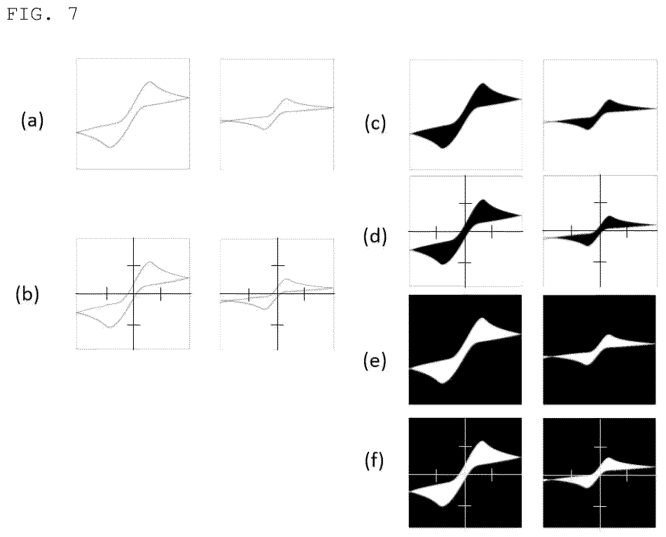

FIG. 7 shows a form of the spectrum image preprocessed according to the present embodiment. In this embodiment, a spectrum image obtained by measuring the sugar content of a beverage containing sugar using a blood sugar sensor has been used.

FIG. 7(a) shows a basic measurement spectrum image, and FIG. 7(b) shows a form of the basic spectrum image with the x-axis and y-axis marked. FIG. 7(c) shows a form in which the internal area has been filled in the basic spectrum image, and FIG. 7(d) shows a form in which the internal area has been filled and axis-marked in the basic spectrum image. FIG. 7 (e) shows a basic spectrum image in which the internal filling has been reversed and the external filling has been performed. FIG. 7(f) shows external filling and axis marking in the basic spectrum image.

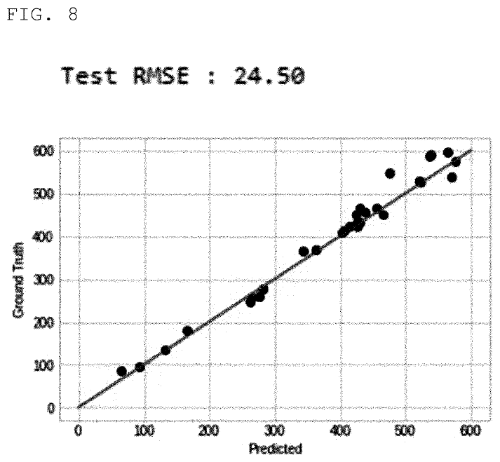

FIG. 8 shows the sugar predicted by the analysis model by measuring the actual sugar of multiple fruits measured using a blood sugar sensor, preprocessing the measured spectrum image as shown in FIG. 7(c), creating an analysis model by learning the preprocessed spectrum image with an artificial intelligence and then inputting the test sample to the analysis model. Referring to FIG. 8, it can be seen that the sugar (ground truth) measured by the actual blood sugar meter and the sugar (predicted) predicted by the analysis model appear almost similar and that the accuracy of prediction has increased compared to FIG. 6.

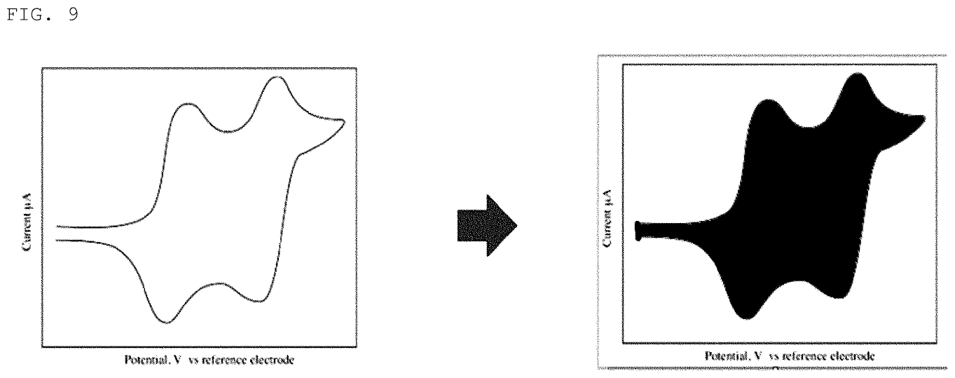

FIGS. 9 to 18 are diagrams showing examples of preprocessing spectrum images for various measurement spectra.

FIG. 9 is a diagram showing image preprocessed by internal-filling for a measurement spectrum image with a complex form.

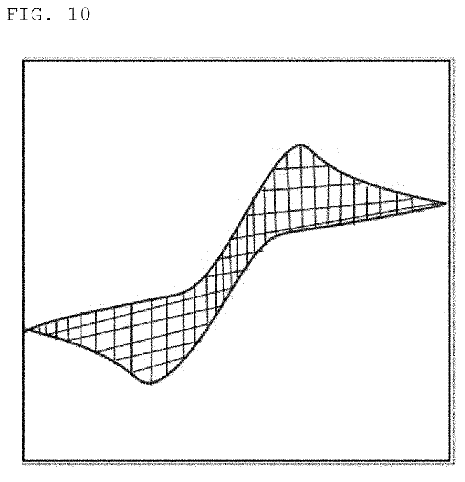

FIG. 10 is a diagram showing image preprocessed by filling the inside of the measured spectrum image in a hatched form.

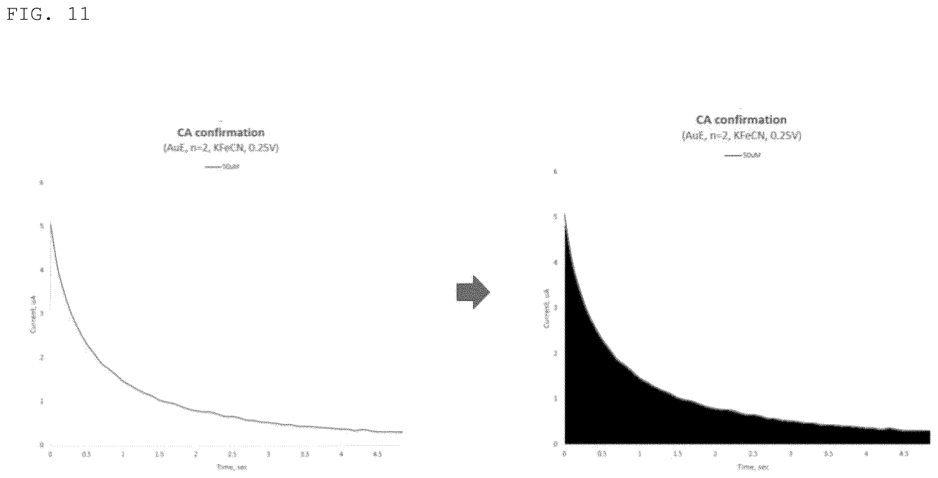

FIG. 11 shows a form preprocessed for the chronoamperommetry spectrum image. Although this spectrum image does not clearly distinguish between the inner and outer areas, it is possible to fill only a partial area of the space divided by the graph by limiting some areas of the spectrum image.

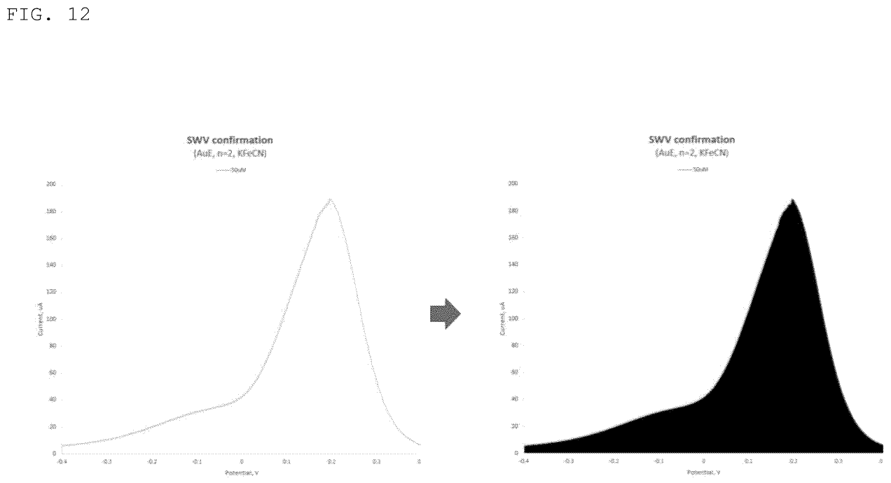

FIG. 12 shows the preprocessed form of the square wave voltammetry spectrum image.

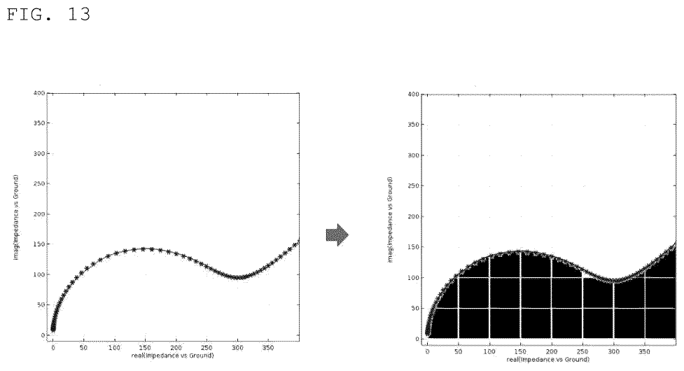

FIG. 13 shows the preprocessed form of the impedance spectrum image.

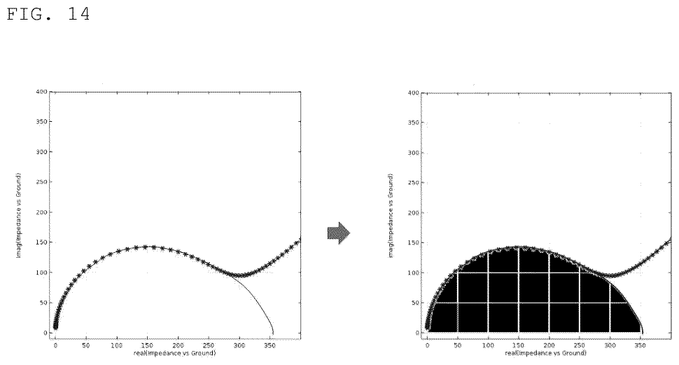

FIG. 14 is a preprocessed form of the impedance spectrum image. In this embodiment, the corresponding area can be filled for each circuit of the equivalent model.

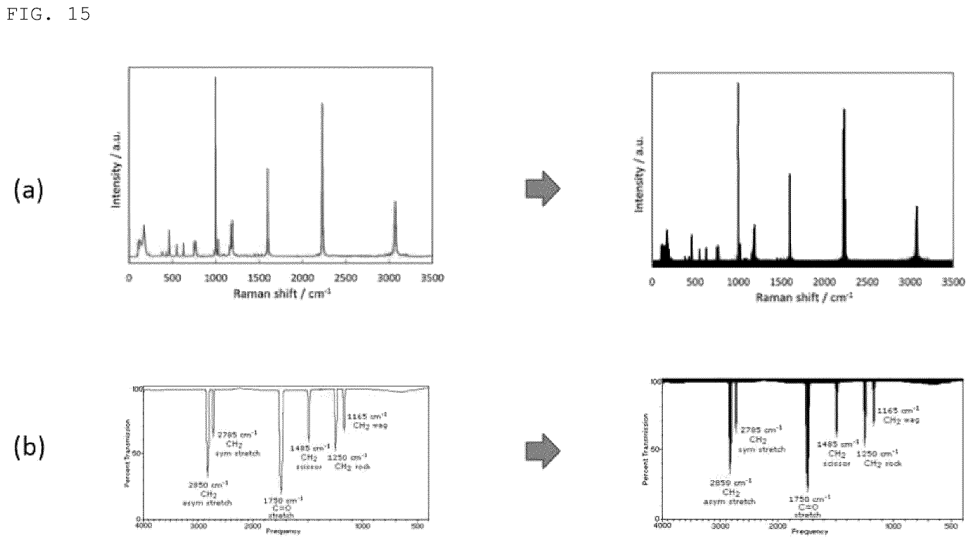

FIG. 15 shows a preprocessed image of other spectroscopic data. FIG. 15 (a) is a Raman analysis graph, and FIG. 15 (b) is a diagram showing image pre-processing performed on the IR analysis graph.

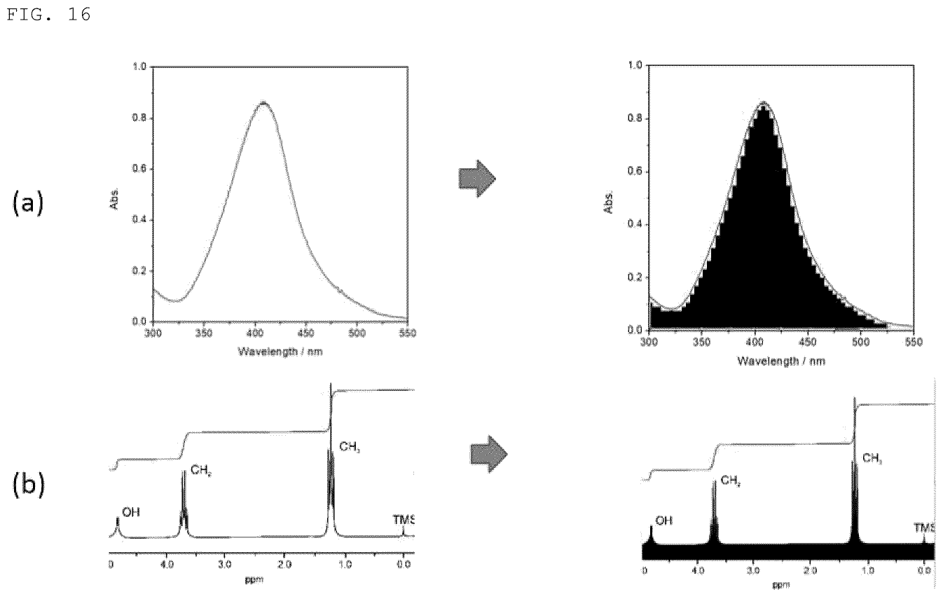

FIG. 16 is a preprocessed image of another spectroscopic data. FIG. 16(a) is a UV measurement graph, and FIG. 16 (b) is a diagram showing image pre-processing performed on the NMR measurement graph.

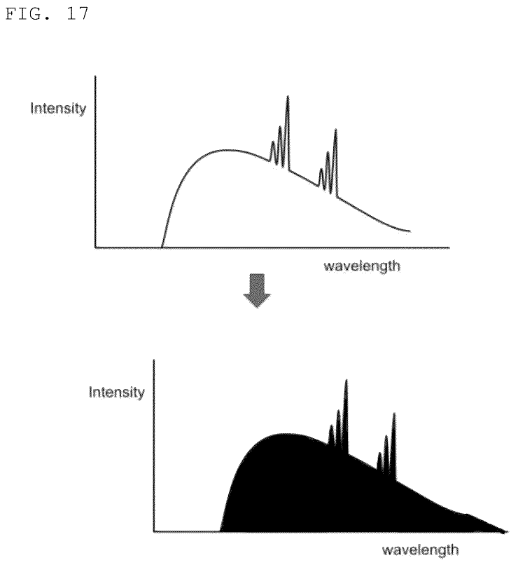

FIG. 17 is a preprocessed form of an X-ray spectroscopic data image.

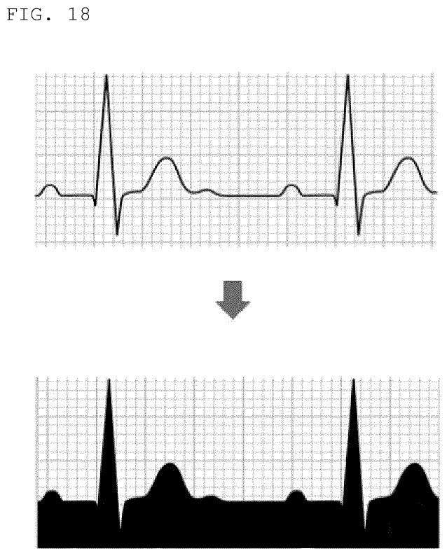

FIG. 18 is a preprocessed form of image for other biosignal spectrum. In this embodiment, the biosignal spectrum may include ECG, EMG, EEG, EKG, etc.

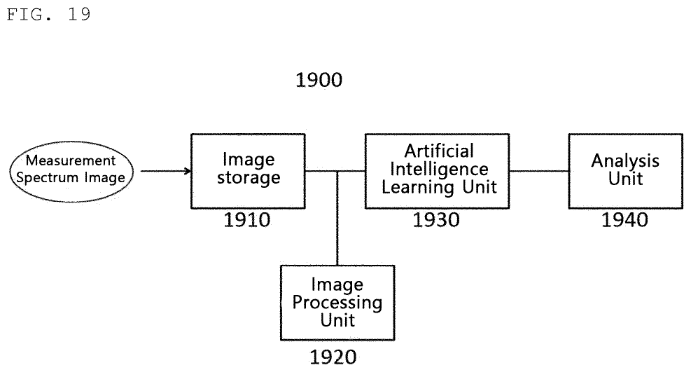

FIG. 19 is a configuration diagram of an apparatus for analyzing a measured spectrum according to another embodiment of the present invention.

Referring to FIG. 19, an apparatus for analyzing a measured spectrum according to the present embodiment may comprise a storage unit 1910 for storing a measured spectrum image, an intelligent learning unit 1930 for creating an analysis model by learning the spectrum image stored in the storage unit with an artificial intelligence and an analysis unit 1940 for predicting the feature of a test sample of the analyte by inputting the spectrum image of the test sample to the analysis model

The storage unit 1910 for storing the measured spectrum image may store the spectrum image for the analyte. In this embodiment, the measurement of the analyte may include various measurements, including electrochemical and physical measurements. For example, when measuring blood sugar in blood, a cyclic voltagram can be obtained using cyclic voltammetry. Here, obtaining the spectrum for the analyte is not limited to thereto and may be obtained in various ways.

The artificial intelligence learning unit 1930 can create an analysis model by learning the spectrum image with an artificial intelligence. Various machine learning techniques such as PCA analysis, SVM, and gradient boosting may be used to learn the spectrum image with the artificial intelligence. In the present embodiment, a convolution neural network (CNN) analysis may be used. The artificial intelligence learning unit 1930 may perform a feature-mapping on the inputted spectrum image using the CNN, and connect the mapped data to a fully connected layer.

The analysis unit 1940 can predict the feature of the spectrum image of the test sample using the analysis model of the artificial intelligence learning unit. The feature of the analyte may be predicted by applying the measured spectrum of the test sample of the analyte whose feature are to be measured to the analysis model. The feature of the analyte that can be predicted may include various feature, including electrical, chemical, and physical feature.

The measurement spectrum analysis device according to the present embodiment may further include an image processing unit 1920 for preprocessing the spectrum image before the learning.

In general, the measured spectrum image can be expressed as a linear graph. When the feature mapping is performed by learning the spectrum image with the artificial intelligence, all areas other than the linear area of the spectrum image are recognized as white, so there is a limitation to extract the features from the image. The preprocessing of the spectrum image can be performed to compensate for the limitation that may appear during learning of the measured spectrum image with the artificial intelligence.

The image processing unit 1920 for preprocessing the spectrum image can perform the preprocesses by drawing an axis on the spectrum image or filling or inverting a partial area of the space divided by the spectrum line in the spectrum image. In this way, when the spectrum image is preprocessed and learned with the artificial intelligence instead of learning the spectrum image as it is, the learning efficiency for the spectrum image is improved, making it much more efficient to extract the feature within the image.

The measurement spectrum analysis device according to this embodiment can also be constructed by a hybride model with an image processing technique for convolutional-processing the spectrum image, several more advanced transformer image processing techniques, and machine learning techniques such as Decision tree, SVM and Boosting that learn a basic tabular data which generates each model image learned from various data.

Although the present invention has been described above with reference to preferred embodiments, those skilled in the art will understand that various modifications and changes can be made to the present invention within the scope of the present invention as recited in the following patent claims. For example, the analytes to be measured and measurement objects, artificial intelligence learning model, etc. may be changed in various ways.

1. A method of analyzing a measured spectrum image comprising the steps of:
obtaining a measured spectrum image;
creating an analysis model by learning the spectrum image with artificial intelligence; and
predicting the feature of a test sample of an analyte by inputting the spectrum image of the test sample to the analysis model.

2. The method according to claim 1, further comprising a step of preprocessing the spectrum image before creating the analysis model.

3. The method according to claim 2, wherein the step of preprocessing of the spectrum image includes drawing an axis on the spectrum image.

4. The method according to claim 2, wherein the step of preprocessing of the spectrum image includes filling or inverting a partial area of the space divided by a spectrum line in the spectrum image.

5. The method of claim 1, wherein the step of creating the analysis model includes feature-mapping the inputted spectrum image to a convolutional neural network (CNN); and connecting the mapped data to a fully connected layer.

6. An apparatus for analyzing a measured spectrum comprising:
a storage unit for storing a measured spectrum image;
an artificial intelligence learning unit for learning the spectrum image stored in the storage unit with an artificial intelligence to thereby create an analysis model; and
an analysis unit for predicting the feature of a test sample of an analyte by inputting the spectrum image of the test sample to the analysis model.

7. The apparatus according to claim 6, further comprising an image processor for preprocessing the spectrum image before the learning.

8. The apparatus according to claim 7, wherein the image processing unit draws an axis on the spectrum image.

9. The apparatus according to claim 7, wherein the image processing unit fills or inverts a partial area of the space divided by the spectrum line in the spectrum image.

10. The method of claim 6, wherein the learning unit for creating the analysis model includes a mapping unit for feature-mapping the inputted spectrum image to a convolutional neural network (CNN); and a classification unit for connecting the mapped data to a fully connected layer.
